(19) United States
(12) Patent Application Publication
(10) Pub. No.: US 2006/0040406 A1
(43) Pub. Date: Feb. 23, 2006

(54) Narcotic analgesic tracking system

(76) Inventors: David Schneider, Louisville, CO (US); Paul Sargeant, Broomfield, CO (US); Joseph Carpenter, Golden, CO (US)

Correspondence Address: MARSH FISCHMANN & BREYFOGLE LLP, Suite 411, 3151 South Vaughn Way, Aurora, CO, 80014, US

(21) Appl. No.: 10/890,823

(22) Filed: Jul. 14, 2004

Related U.S. Application Data
(60) Provisional application No. 60/545,625, filed on Feb. 17, 2004.

(51) Int. Cl. G01N 33/558

(52) U.S. Cl. 436/514

(57) ABSTRACT

A device and method for testing the contents of a sample of a controlled substance to confirm the presence or absence of the controlled substance within that sample. In one implementation, the device and method utilize antibodies designed to interact with one or more anesthetics, e.g., opioid analgesics. The antibodies may be tagged (e.g., attached to a colored marker) to allow for optically identifying the occurrence of an antibody reaction with an antigen (e.g., associated with a given anesthetic) within a sample. The antibody/antigen reaction may be implemented in connection with a number of different devices, including fluid drop testers and thin layer chromatographic systems. In addition to the presence of a anesthetic in a given sample, the inventive device may identify the concentration of the anesthetic in order to identify diluted samples. In this manner, handling of anesthetics in connection with medical procedures, including transfer of custody and wasting, can be better monitored, for example, to inhibit diversion of anesthetics and reduce associated medical and legal concerns.

### RELATED APPLICATION

This patent application claims priority from U.S. Provisional Patent Application No. 60/545,625, which was filed on Feb. 17, 2004, and is entitled “DEVICE FOR THE CONFIRMATION OF DISPOSAL OF POST-OPERATIVE WASTE NARCOTIC ANALGESIC.” The entire disclosure of U.S. Provisional Patent Application No. 60/545,625 is incorporated herein by reference.

### FIELD OF THE INVENTION

The present invention relates generally to a test device and associated methodology for monitoring the handling of a controlled substance, e.g., by determining at least the presence of a predetermined substance in a liquid sample. More particularly, the present invention is directed to determining the presence and, preferably, the concentration of an analgesic substance in a liquid sample.

### BACKGROUND OF THE INVENTION

There are approximately 30 million surgical procedures performed annually in the United States in addition to 90 million emergency room visits. In nearly all of the surgical procedures and many of the emergency room visits, analgesics are utilized. Such analgesics include opioid analgesics such as fentanyl, sufentanyl, morphine and dilaudid. While effective in reducing patient pain and discomfort, many of these substances are also highly addictive. Due to their additive nature, dispersion of these substances is typically controlled. In general, these controls prevent widespread patient abuse due in part to the difficulty in procuring these substances. However, some medical personnel such as physicians and particularly anesthesiologists require ready access to these substances as part of their day-to-day duties. Due to this access and the stresses inherent in their jobs, such medical personnel are more likely than the general population to use and become addicted to these substances.

Some estimates place anesthesiologist analgesic dependence at 0.10 percent and the incidence of resident analgesic dependence at 0.40 percent. While appearing low on a percentage basis, it will be appreciated that in the United States alone this amounts to a significant number anesthesiologists and residents who each may perform multiple procedures in a given working day. Coupled with the nature of their work, the consequences of analgesic abuse by anesthesiologists have the potential to be catastrophic. The total personal, social and economic consequences (e.g., liability) of anesthetic abuse can only be estimated, but, in a particular example, the economic impact to a facility at which abusing physicians were resident was estimated to run into hundreds of thousands of dollars of legal and other fees and untold costs in terms of bad publicity and reduced confidence.

To prevent medical personnel from abusing analgesics, a number of controls have been implemented. For instance, analgesic substances for a specific procedure are typically obtained from the hospital dispensary. An official at the dispensary generally records the amount of analgesics and to whom the analgesics are dispensed. However, due to the uncertain nature of many medical procedures (e.g., the length of the procedure, the patient's pain tolerance, etc.) and the desire not to run out of analgesic during a procedure, the dispensed amount of analgesic typically includes a safety margin. That is, the dispensed analgesic includes a surplus amount over a predetermined amount expected to be utilized for a procedure.

The dispensed amount of analgesic(s) is typically provided in one or more sealed vials. The analgesic is often drawn from the vial(s) into a syringe for easy administration to the patient during a procedure. As will be appreciated, unused analgesic cannot be reused for another procedure. Accordingly, such post-operatively/post-procedurally unused analgesic is routinely disposed of. That is, at the conclusion of the procedure, any unused analgesic (e.g., in a syringe and/or partial vials) is “wasted” by the medical personnel in the presence of another person. The disposal of residual, unused analgesic is generally recorded in a procedure log and witnessed by a member of the medical center staff.

This recording is an event mandated by regulatory bodies in an attempt to inhibit misuse of the analgesics. However, the analgesics are typically clear and water-like. In this regard, there are numerous instances where an abusing anesthesiologist has adulterated unused anesthetic with saline (i.e., before or after a medical procedure) in order to secure a supply of drug for later abuse and fraudulently confirmed his disposal of anesthetic wastage—a process known as “diversion.” The diversion process comprises an important source of drugs for abusing physicians.

### SUMMARY OF THE INVENTION

The inventors of the present invention have recognized that a combination of circumstances appear to lead to the high incidence of drug abuse among anesthesiologists compared to other physicians and medical personnel. These circumstances include: 1) the highly addictive properties of surgical analgesics; 2) the widespread and easy availability of analgesics to anesthesiologists as a group; 3) the job-related stresses of anesthesiologists; and 4) the lack of stringent controls/monitoring of the handling of analgesics including transfers of custody and disposal of unused analgesics. The inventors have further recognized that while little can be done about the first three circumstances, it is possible to better monitor the handling of analgesics, from the supply of analgesics from the hospital dispensary through patient administration and ultimately to the disposal of “waste” quantities. In particular, the inventors have recognized that the current disposal system is open to abuse since no positive confirmation is obtained that actual analgesic or non-diluted analgesic is being disposed of or otherwise transferred. Accordingly, the inventors have recognized that confirming the contents of a sample upon wastage or other transfer may further reduce the availability of these substances and possibly reduce the abuse of such substances. It will be appreciated that the invention, while having particular advantages in the context of verifying the wasting of narcotic analgesics, may be used to monitor the handling of other controlled substances, especially those that can be readily adulterated for purposes of diversion and those that include antigens.

In this regard, the present invention is directed to a device and method for testing the contents of a putative sample of a controlled substance, such as a narcotic analgesic, to confirm the presence or absence of the controlled substance within that sample. Importantly, it has been realized that such analgesics, including opioid analgesics, typically include antigens. That is, these substances trigger an immune response that results in the production of an antibody as part of the body's defense against infection and disease. Accordingly, such an antibody may be produced for use in identifying a matching antigen within the substance under analysis. These antibodies may be tagged (e.g., combined or otherwise associated with a colored marker) in such a manner that allows for physically identifying the occurrence of an antigen/antibody reaction in a sample.

Utilization of such antibody/antigen reaction may occur in a number of different assay test systems. For instance, such assay test systems may involve drop tests wherein drops of a putative sample are dropped into a reactant liquid that includes antibodies and/or an optical indicator. For example, if a matching antigen substance is present within the drop sample, the liquid may turn a characteristic color. Alternatively, thin layer chromatographic systems in which a solvent or other liquid moves across a thin, flat, absorbent medium may also be utilized. Typically, the absorbent medium is inoculated in one or more places with one or more types of antibodies that will react with antigens within a liquid sample. Such systems may employ what may be termed test strips and/or dipsticks, though the term test strips is used below for convenience. It will be appreciated that the term encompasses non-liquid media, of any suitable configuration, that support the testing materials.

The test strips may include antibodies that are mobile (e.g., in the presence of a liquid sample) and linked to an indicator agent such as dyed latex, a colloidal medial sol, or radioisotope. Once a liquid sample is applied to the test strip, the liquid sample and mobile antibodies may then be applied (e.g., through capillary action or mixing within a fluid media) to an indicator (e.g., chromatographic) medium containing a zone of immobilized antibodies for an antigen of interest. When the sample including the antigen of interest is provided, those antigens bond with the mobile antibodies and the associated indicator agent as well as the immobilized antibodies of the indicator medium. That is, the indicator agent of the mobile antibodies may be bound to the immobilized antibodies (i.e., by the antigens) in a predetermined area of the strip thereby indicating the presence of the antigens within the sample.

According to one aspect of the present invention, an assay device for use in monitoring handling of analgesics in connection with a medical procedure is provided. For example, the device may be used to monitor transfer events such as changes in custody or wasting of the analgesic. The assay device includes a test medium having an anti-analgesic antibody disposed therein. In addition, the device includes an optical agent also disposed within the test medium for providing an optical indication relating to a contact of an analgesic corresponding with the analgesic antibody.

Various refinements exist of the features noted in relation to the subject first aspect of the present invention. Further features may also be incorporated in the subject first aspect of the present invention as well. These refinements and additional features may exist individually or in any combination. For instance, the test medium may comprise a liquid medium into which a sample of a putative analgesic may be applied. Alternatively, the test medium may comprise a test strip operative to provide an optical indication of the presence or absence of an analgesic within a sample applied to that test strip. The test strip may, in a further embodiment, be formed as a lateral flow dipstick that allows for liquid samples to spread therethrough via capillary action. In this regard, nitrocellulose materials may be utilized. It will be appreciated that such nitrocellulose materials provide a suitable medium into which antibodies may be impregnated. In any case, use of such a test strip may allow for quickly and conveniently testing a liquid sample.

The anti-analgesic antibody disposed within the test medium may be tailored for a particular analgesic or a family of analgesics. In this regard, it may be possible to provide an antibody that is operative to indicate the presence of one or more of a family of analgesics, such as opioid analgesics (e.g., fentanyl, sufentanyl, morphine and dilaudid). In any event, the antibody is preferably operative to bind to a particular antigenic binding site of an analgesic as well as bind to a marker such that an optical indication of the presence of the analgesic may be provided. Further, it will be appreciated that more than one type of antibody may be included within the test medium. In this regard, the test medium may include a plurality of antibody types such that the assay test is operative to identify the presence of a plurality of different analgesics.

The optical agent disposed within the test medium may be any agent that is operative to indicate, e.g., optically, the attachment of the anti-analgesic antibody or antibodies. For instance, in one embodiment latex beads covered with anti-analgesic antibodies are utilized. These latex beads may be colored such that when a group of these beads are concentrated in an area (e.g., on a test strip) due to an antigen/antibody reaction the concentrated latex beads may provide a visual indication of the presence of analgesic within a tested liquid sample. Further, it will be noted that in some embodiments, different colored optical indicators may be utilized. For instance, when utilizing a lateral flow test strip where a liquid is operable to pass through the test strip by a capillary action, different sections of the strip may include different colors. Likewise, these different colored optical indicators may be utilized for indicating the presence of different analgesics and/or the concentration of such analgesics. Additionally or alternatively, the concentration of the analgesic may be indicated by the concentration of single color indicators on the test medium, resulting in shading that can be calibrated to different analgesic concentration levels.

When the assay test comprises a test strip, the test strip may incorporate a test band that provides an optical identification of the presence and/or type of analgesics within a sample. Such a test strip may further incorporate a control band that provides a visual indication that the test strip has been utilized. For instance, such a test band may include anti-species antibodies that are operable to attach the antibodies, which are matched to a particular analgesic. In this regard, anti-species antibodies may attach to antibodies having optical indicators such that the optical indicators (e.g. latex beads) may attach to the control band to provide an optical indication that the test strip has been utilized. This indication may be provided even though no analgesic is within the liquid sample. For example, the control band may utilize an antibody/antibody reaction as opposed to an antibody/antigen reaction.

In addition to providing an indication that an analgesic is present or absent from a test sample, the assay test may further provide an indication of the concentration of the analgesic within the sample. For instance, in one embodiment where an optical indication is provided by a color, a color chart may be provided that allows for determining the concentration of an analgesic within a sample. For instance, the shade of the resulting color may provide this indication. Other methods may be utilized as well.

According to another aspect of the present invention, a method for monitoring the disposal of unused analgesics in connection with a medical procedure performed at a procedure location is provided. The method includes the steps of providing, in proximity to the medical procedure location, a test device for verifying at least the presence of an analgesic in a test sample. Accordingly, a putative, unused analgesic sample is provided for verification. The test device is used to test the putative, unused analgesic sample so as to provide an indication concerning the analgesic content of the test sample. Information regarding the disposal of the unused analgesic, based on the indication, may be recorded.

The test device may include an indicator (e.g., associated with an assay test as described above) that provides an optical indication regarding the presence of the analgesic. Preferably, the indicator will further provide an indication regarding the concentration of the analgesic in the sample. In this regard, it will be appreciated that diluted samples that contain a reduced amount of the analgesic may be identified, thus inhibiting diversion by dilution.

Using the test device may involve contacting at least a portion of the putative, unused analgesic sample with an anti-analgesic antibody. This may entail dropping fluid into a liquid medium or, contacting an absorbent test strip with a portion of the sample or immersing such a test strip within a sample. Once the test device has been in contact with the putative sample, the test device can be read to determine the absence, presence, and/or concentration of analgesic within the sample. In some instances, this may entail simply identifying a marker within the test medium (e.g., +/−). Alternatively, this may comprise identifying a color within the test medium and/or comparing the color to a reference chart for determining concentration. It will be further appreciated that, depending on the test medium, a concentration chart may be incorporated into the test device for verifying without necessitating comparison to a chart.

According to another aspect of the present invention, a pre-packaged product for use in connection with monitoring the handling of analgesics is provided. The product includes a package having at least one dosage of a particular analgesic for use in a medical procedure and at least one assay device, enclosed within the package, for use in verifying the presence of the analgesic within a liquid sample. The test device may include a test medium having an antibody operative to attach to the particular analgesic. Additionally, the device may include a optical agent disposed within the test medium for providing an optical indication relating to the contact of the analgesic and the antibody. The prepackaged product may further include instructions and/or charts for use assay device to identify positive, negative and/or inconclusive test results and/or analgesic concentrations. Furthermore, the packaging may include a number of dosages of analgesic and a number (e.g., corresponding to the number of dosages) of assay devices. As will be appreciated, by developing an assay device (i.e., test medium) for a particular analgesic, and distributing that assay device with the analgesic, a facility purchasing the analgesic may better control its use and inhibit its diversion, thereby reducing medical and legal concerns.

According to another aspect of the present invention, a method for monitoring the handling of a controlled substance is provided. The method comprises the steps of contacting a test specimen including antibodies specific to the controlled substance with a putative sample of the controlled substance for a predetermined antibody binding period. In this regard, it will be noted that the binding period may depend upon the type of medium (e.g., liquid, absorbent, etc) in which the antibodies are disposed. After the predetermined antibody binding period, the test specimen may be removed from the sample such that information regarding any controlled substance in the sample may be determined. Such information may include the presence or absence of the controlled substance. Further, when the controlled substance is present, that information may further include the concentration of controlled substance within the sample. A corresponding inventive test device includes a support medium and antibodies, associated with the support medium, that are specific to the controlled substance under consideration.

According to another aspect of the present invention, a method for monitoring the handling of analgesics within a medical facility is provided. The method includes dispensing a known quantity of a given analgesic and in conjunction with dispensing, providing an assay device adapted to detect that particular analgesic. This assay device may be provided to the same individual who received the analgesics, or, to a second individual who will be assisting with and/or present during a given medical procedure in which the analgesic will be administered. A sample of the analgesic may be tested in connection with any transfer event. For example, after the medical procedure, a putative sample of an unused portion of the analgesic may be contacted with a test medium of the assay device. Accordingly, information regarding the analgesic in the sample may be determined from the test medium.

According to another aspect of the present invention, an automated system for monitoring handling of analgesics is provided. The automated system is operative to receive a putative sample of an analgesic for a determination regarding a particular analgesic. The system is further operative to analyze the putative sample and provide an indication of the contents thereof. In this regard, the system may contact a portion of the putative sample with a test medium (e.g., containing anti-analgesic antibodies). After contacting the sample with the test medium, the system may further analyze, e.g., optically, the reaction of the sample and the test medium to identify a concentration of analgesic if present. For example, such analysis may involve using a spectrometer or other optical device to determine a color, shade or other optical property of the test medium after exposure to the sample. A processor can then correlate this determination to a particular analgesic/concentration. It will be appreciated that such automation or partial automation provides a number of potential advantages including increased accuracy (particularly in identifying concentration) and reduced opportunity for fraudulent manipulation.

### BRIEF DESCRIPTION OF THE DRAWINGS

For a more complete understanding of the present invention and further advantages thereof, reference is now made to the following detailed description taken in conjunction with the drawings, in which:

FIG. 1 shows a test strip in accordance with the present invention;

FIG. 2 shows attachment of antibodies to the test strip of FIG. 1;

FIG. 3 shows a process flow sheet that may be utilized with the test strip of FIG. 1;

FIG. 4 shows an automated system that may be utilized with the test strip of FIG. 1 and

FIG. 5 illustrates a voucher that may be utilized to track handling of a controlled substance in accordance with the present invention.

### DETAILED DESCRIPTION

In the following description, the invention is set forth in the context of a system for monitoring the handling of analgesics at a medical facility. Such handling may involve changes of custody, wasting and other transfer events. As noted above, this is a particularly useful implementation of the invention because: the potential for diversion is great as analgesics are readily adultered and the professionals who handle the analgesics are sophisticated and technically capable of avoiding detection; the temptation for diversion is great as such analgesics are highly addictive, are readily available to health-care professionals and those professionals have highly stressful work environments; and the potential harm of diversion is tremendous because these professionals often monitor one the most critical and perilous aspects of medical procedures, anesthesia, not to mention the harm to facility reputation that may occur. While this application provides significant motivation for the present invention, it will be appreciated that various aspects of the invention have broader applicability, especially with respect to monitoring handling of controlled substances.

In accordance with the present invention, an assay test is provided that can be utilized to detect the presence or absence of a substance in a volume of liquid. More particularly, the assay test can be utilized for detecting analgesics (e.g., opioid narcotics) within a solution. Further, in the event that a suspected substance is detected, the assay test is designed to provide an optical (e.g., visual) indication of the same. As discussed herein, the assay test is directed towards detecting opioid analgesics and in particular fentanyl. However, it will be appreciated that an assay test as disclosed herein may be configured to detect different analgesics and/or a plurality of analgesics.

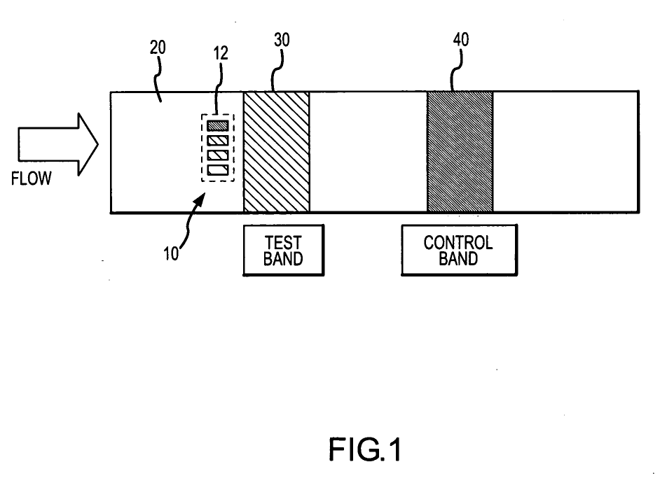

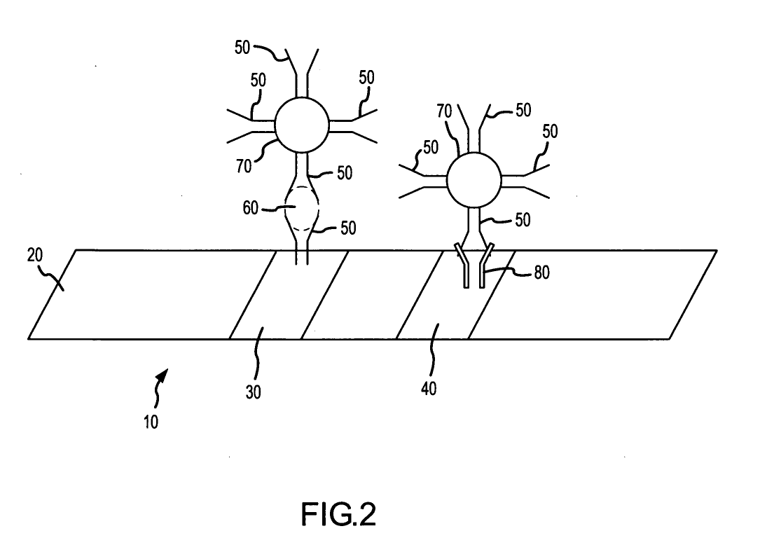

FIGS. 1 and 2 show one embodiment of an assay test strip 10 that is designed to identify the presence fentanyl within a liquid sample. As shown, the test strip 10 includes a nitrocellulose strip 20 that is selectively impregnated with antigen-specific antibodies. As shown, nitrocellulose strip 20 may be termed a lateral flow dipstick. In this regard, the nitrocellulose strip 20 may be dipped into a liquid test sample to allow the liquid to spread through the nitrocellulose strip 20 via capillary action. However, it will be appreciated that the nitrocellulose strip 20 may also be immersed in a test sample if an adequate volume of the test sample is provided.

Disposed on the surface of the test strip 10 are a test band 30 and a control band 40. The nitrocellulose strip 20 is impregnated with antibodies across the width of each band 30, 40. In particular, the nitrocellulose strip 20 is impregnated with a plurality of anti-fentanyl antibodies 50 (only one shown for illustrative purposes) disposed across the width of the test band 30 and a plurality of anti-species antibodies 80 (only one shown for illustrative purposes) disposed across the width of the control band 40. See FIG. 2. These antibodies 50, 80 have a specific binding affinity and selectivity for an antigen of interest. In particular, the anti-fentanyl antibodies 50 have an affinity for fentanyl and the anti-species antibodies 80 have an affinity for the anti-fentanyl antibodies 50.

The bands 30, 40 are designed to provide an optical indication concerning the presence of fentanyl within a liquid test sample that is applied to the test strip 10. In this regard, the surface of the nitrocellulose strip 20 and/or each band 30, 40 further includes a plurality of colored latex beads 70 (only one shown over each band 30, 40 for illustrative purposes) that are each coated with anti-fentanyl antibodies 50. Prior to application of a liquid to the test strip 10, the latex beads 70 are immobilized. However, when a liquid sample is applied to the test strip 10, the latex beads 70 become mobile. In order for either of the bands 30, 40 to change color, or become colored as the case may be, the anti-fentanyl covered latex beads 70 must attach to the antibodies disposed across the width of the bands 30, 40.

In this regard, when the test strip 10 is wetted the anti-fentanyl antibodies 50 disposed on the latex beads 70 may contact the anti-species antibodies 80 within the control band 40 as shown in FIG. 2. Accordingly, the anti-fentanyl antibody 50 and anti-species antibodies 80 will bond, attaching the anti-fentanyl covered latex beads 70 to the control band 40. Accordingly, when enough latex beads 70 become attached to the control band 40, the control band 40 turns the color of the latex beads 70. As will be appreciated, the control band 40 is provided to determine whether the test strip 10 functioned correctly. In this regard, the control strip 40 consists of a band of anti-species antibodies 80, to which anti-fentanyl antibodies will bond irrespective of the presence of fentanyl in the liquid sample applied to the nitrocellulose strip 20. That is, all that is required for the control band 40 to change color is that the test strip 10 be wetted such that the anti-fentanyl covered latex beads 70 to become mobile.

In contrast, for the test band 30 to change color or become colored, fentanyl must be present within the liquid sample applied to the test strip 10. Again, coloration of the test band 30 will result from the attachment of the anti-fentanyl covered latex beads 70. As previously noted, the latex beads 70 include anti-fentanyl antibodies 50 attached to their surface. Likewise, the test band 30 includes a plurality of anti-fentanyl antibodies impregnated within the nitrocellulose strip 20 throughout the width of the test band 30. In order for the latex beads 70 to attach to the test band 30, fentanyl must be present in the liquid sample. As shown in FIG. 2, a fentanyl molecule 60 attaches to a first anti-fentanyl antibody 50 on the surface of the test band 30 and to a second anti-fentanyl antibody 50 on the colored latex beads 70. Accordingly, this effectively binds the colored latex beads 70 to the test band 30. As will be appreciated, if fentanyl is not present within the liquid test sample applied to the test strip 10, the colored latex beads 70 will not bind to the test band 30 indicating that fentanyl is not present within the test sample.

It will be appreciated that variations may exist of the test strip 10 discussed above. For instance, the test band 30 and control band 40 may utilize latex beads 70 of different colors such that a positive result of each is more easily recognized. Likewise, though discussed as utilizing a dyed latex beads 70 to provide an optical indication, it will be appreciated that additional materials may be utilized to provide an optical or other indication of the presence of fentanyl or other analgesics within a test sample. Such materials include, without limitation, colloidal metal sol, and radioisotopes.

When a liquid sample is applied to the test strip 10, a number of outcomes are possible. For instance, if the test band 30 and control band 40 both test positive (i.e., turn color indicating that the latex beads 70 are bonding to the bands 30, 40), it may be assumed that fentanyl is present within the sample and the assay test worked correctly. Alternatively, the test band 30 may be positive (i.e., colored), while the control band 40 may be negative (i.e., uncolored), indicating an abnormal result, or failure of the assay test. Finally, the test band 30 may be negative and the control band 40 may be positive, thereby indicating the test worked and resulted in a suspicious result wherein little or no fentanyl is present within the liquid sample. In addition, as discussed above, the shading of the test band 30 may provide an indication of the concentration of the fentanyl. In this regard, an optional shading key 12 may be provided on the strip 10 to facilitate correct recording of the indicated concentration. In this manner, diversion by dilution is discouraged.

To utilize the test strip 10 in the confirmation of wastage, it may be preferable that the test strip 10 be incorporated into existing medical procedure workflows. For instance, it may be desirable that such a test strip 10 be provided with the fentanyl 60 (or other analgesic as the case may be) upon purchase. That is, if the analgesic is packaged in a vial, a test strip 20 may be provided for each such vial. Accordingly, when a physician is dispensed the analgesic from the dispensary at the hospital, they may be provided with at least one test strip 10 for use in testing any kept wastage. Alternatively, and/or in addition, such test strips 10 may be provided separately and kept in proximity to the location where said analgesics are utilized. More particularly, such test strips 10 may be kept where unused analgesics are typically disposed of.

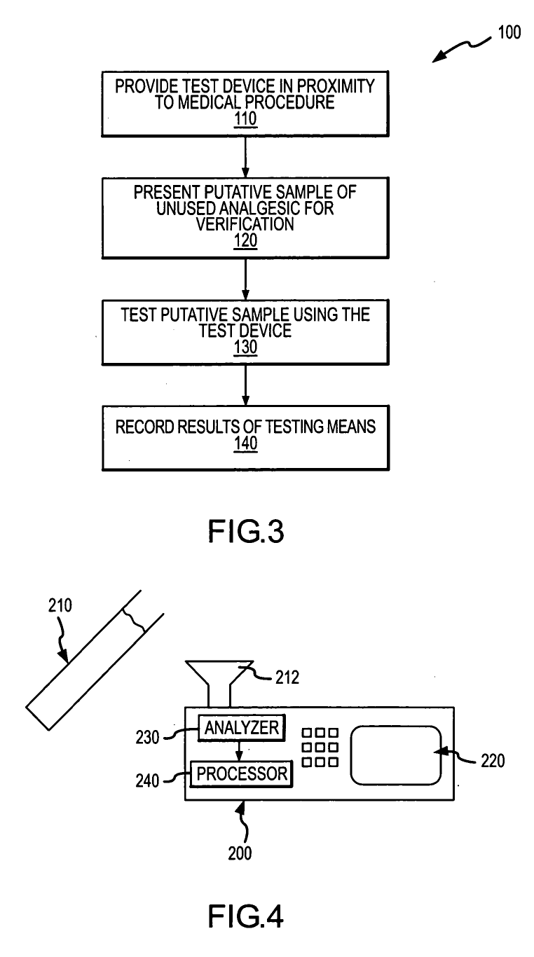

A possible workflow compatible process (100) for use in confirming the contents of analgesic wastage is shown in FIG. 3. As shown, the process initially includes procuring (110) a quantity of analgesic from a medical facility dispensary. The procuring step (110) may be witnessed by one or more individuals and/or recorded to identify who receives the analgesic and the amount of analgesic received. This analgesic may then be transported (115) to the location where a medical procedure is to be performed. Such locations may include, without limitation, doctors offices, operating rooms, and emergency rooms.

During a medical procedure, a portion of the analgesic may be administered (120). In this regard, one or more analgesic containers (e.g., vials) may be opened and administered (120). The opening and/or administration of the analgesic may be recorded. Once the medical procedure is completed, any remaining analgesic may be transported (125) to a “wasting” area. In this regard, a person in charge of the analgesic (e.g., an anesthesiologist) may be accompanied to the wasting area. A person accompanying the anesthesiologist may obtain (130) a test device (e.g., the test strip 20 or other assay testing means) provided in proximity to the wasting area for verifying the presence of an analgesic in the putative remaining analgesic (i.e., putative sample). Such testing means may be kept in a secured location (e.g., a locked cabinet) to prevent possible tampering.

The test device may then be prepared (135) to test the putative sample. This may entail opening a sealed package in which the test device is contained. As will be appreciated, the test device may be disposed within a sealed package to preserve the reactive agents (e.g., antibodies) of the test device and/or to further prevent tampering. Accordingly, the preparing of the test device may be witnessed and recorded. The putative sample may then be presented for verification. Using the test device, the putative sample may be contacted (140) with the test device (e.g., manually or in an automated process) to provide an indication concerning an analgesic content of the putative sample. Accordingly, an optical indicator of the test device may be read and the results may be recorded (145). Further, it will be noted that the test device may be preserved (150) for evidentiary purposes and/or further testing if so desired. In this regard, the test device may be provided as part of a kit including a container in which the test device and/or any remaining portion of the putative sample may be sealed for further analysis. For instance, if the testing device is inconclusive or negative as to the presence of analgesic within the sample, the test device may be preserved (150) for further analysis. As will be appreciated, by providing such a test device in proximity to the medical procedure, use of the test device may be incorporated into the medical procedure without substantially changing existing procedures.

FIG. 5 illustrates a controlled substance voucher 500 that may be used in accordance with the present invention. The voucher is filled out by facility personnel in connection with one or more medical procedures and includes a number of conventional fields such as identification fields 502 and inventory tracking fields 504, 506 and 510. these conventional tracking fields attempt to track controlled substances by identifying the amounts and substances withdrawn from the dispensary, the amounts and manner of disposition (e.g., used, wasted, etc.), and the amounts returned. As noted above, such conventional tracking allows for diversion by motivated personnel.

In accordance with the present invention, additional test fields 508 are provided for recording the results of tests as described above for verifying the content of analgesics used, wasted and/or otherwise transferred. For example, facility policy may require that the analgesic content of a sample be tested and the results recorded in connection with administration of analgesics to a patient, or only in connection with wastage events. For example, a test strip may be used as described above and the results (e.g., positive for analgesics or a concentration value) may be recorded in fields 508 together with witness initials. These results may be entered manually or the control band and/or test band, or the entire surface of the test strip including the control and test bands, may be attached to the voucher 500. For example, the test band may be provided as a sticker, optionally with a printed serial number or other authentication information, that can be removed from the test strip and applied to the voucher 500. This provides reduced opportunities for fraud and improves tracking, as well as providing a reliable evidentiary record for use in disciplinary or legal proceedings.

The illustrated voucher 500 further includes novel usage rate fields 512, for example, for tracking procedure times, analgesic usage and usage-per-time data. It will be appreciated that diversion may be accomplished, for example, by adulteration of waste or administered analgesics, or by exaggeration of properly used analgesics. Adulteration is addressed by the test strip process and fields 508. Exaggeration is addressed by fields 512. Specifically, by tracking the amount of analgesics used by particular personnel in relation to procedure time lapsed, individuals associated with suspicious usage patterns can be identified. For example, such individuals may then be subjected to close scrutiny regarding controlled substance usage. Moreover, honest but questionable medical usage of controlled substances may be identified in this manner.

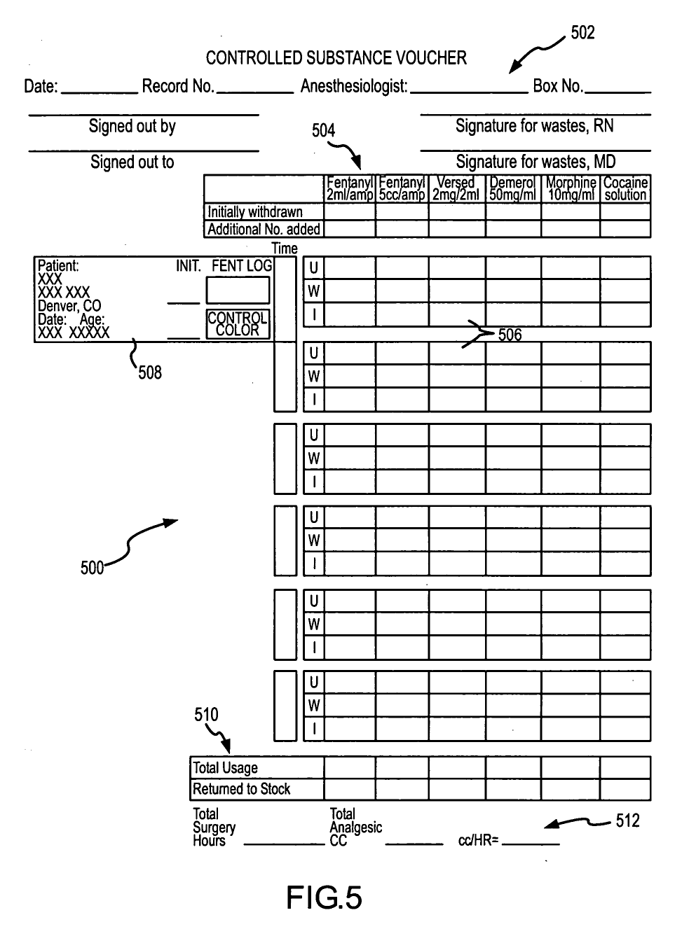

Though discussed above as providing a test band 30 and control band 40 designed to provide an optical or other indication for visual inspection, it will be appreciated that the optical indication could also be conveniently read by an automated detection system. For instance, as shown in FIG. 4, a putative sample may be provided to an automated system 200 where the automated system 200 is operative to receive a putative sample of unused analgesic 210 via a sample port 212 and use that sample 210 to wet a test strip—or otherwise apply the sample to a test medium that includes the anti-fentanyl antibodies. Once so applied, an analyzer 230, which may include, for example, an optical detector such as a spectrometer or different wavelength LEDs associated with an optical sensor, is operative in conjunction with processor 240 to analyze the results of the application and may provide an output relating to the presence, absence and/or concentration of analgesic within the sample 210. Such an output may be provided on, for example, an attached display 220. Alternatively, the system may be operative to receive a pre-wet test strip 10 and analyze the results. In this regard, the system 200 may be operative to determine a relative accumulation of the latex beads 70 in the test band 30 and/or control band 40. This may be performed by suitable instrumentation, minimizing the need for human attention. Furthermore, utilization of such instrumentation may allow for determining, inter alia, the concentration of analgesic within the sample. Determining such a concentration may be done in any appropriate way. For example, this may include utilization of a colorimeter.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1-6. (canceled)

7. A device for use in monitoring a disposal of unused analgesics in connection with a medical procedure performed at a procedure location, comprising:
a support medium;
an anti-analgesic antibody supported by said support medium; and
an optical agent, for providing an optical indication relating to a contact of an analgesic and said anti-analgesic antibody.

8. The device of claim 7, wherein said support medium comprises a fluid.

9. The device of claim 7, wherein said support medium comprises an absorbent material.

10. The device of claim 9, wherein said absorbent material comprises a dip-stick.

11. The device of claim 10, wherein said dip-stick includes a first area containing anti-analgesic antibodies.

12. The device of claim 11, wherein said dip-stick includes a second area containing anti-species antibodies.

13. The device of claim 7, wherein said optical agent comprises a plurality of colored markers each including at least one interconnected anti-analgesic antibody.

14. The device of claim 13, wherein said colored markers comprises latex beads.

15. The device of claim 7, wherein said optical agent provides a colored indication relating to a contact of an analgesic and said anti-analgesic antibody.

16. The device of claim 15, further comprising:
a color chart for use in determining a concentration of an analgesic in a sample, based on said colored indication.

17-38. (canceled)

39. A device for use in monitoring a disposal of unused analgesics in connection with a medical procedure performed at a procedure location, comprising:
a fluid port for receiving a putative sample of an analgesic;
a reactant liquid disposed within said fluid port, said reactant liquid including:
an anti-analgesic antibody adapted to bind to said analgesic; and
an optical indicator agent adapted to bind to said analgesic; and
a test medium impregnated with said anti-analgesic antibodies, wherein upon contacting a mixture of said reactant liquid and said sample of analgesic, said test surface provides an indication of a concentration of said putative-sample of said analgesic.

40. The device of claim 39, wherein said test medium provides one of a positive indication and a negative indication of a presence of said analgesic in said putative sample.

41. The device of claim 39, wherein said test medium further comprises:
anti-species antibodies for binding with said anti-analgesic antibodies.

42. The device of claim 39, wherein said device is operative to contact said test medium with a mixture of said reactant liquid and said putative sample of said analgesic.

43. The device of claim 39, wherein said analgesic is fentanyl and wherein said anti-analgesic antibodies are anti-fentanyl antibodies.

44. The device of claim 39, wherein said optical indicator agent comprises anti-analgesic antibody attached to a colored marker.
